(19) United States
(12) Patent Application Publication
(10) Pub. No.: US 2023/0255546 A1
(43) Pub. Date: Aug. 17, 2023

(54) PREPARATION OF NANO SILVER/DUAL MODIFIED CHITOSAN ANTIBACTERIAL HYDROGEL DRESSING WITH DISCOLORATION EFFECT FOR WOUND INFECTION JUDGEMENT AND HYDROGEL DRESSING PREPARED BY THE SAME

(71) Applicant: ZHEJIANG UNIVERSITY, Hangzhou (CN)

(72) Inventors: Xiaoqiang JIN, Hangzhou (CN); Zhaoming YE, Hangzhou (CN); Xiaojiong BAO, Hangzhou (CN); Huihong JIANG, Hangzhou (CN); Wangsiyuan TENG, Hangzhou (CN); Zhijun YU, Hangzhou (CN)

(21) Appl. No.: 18/306,986

(22) Filed: Apr. 25, 2023

Related U.S. Application Data
(63) Continuation of application No. PCT/CN2022/117686, filed on Sep. 8, 2022.

(30) Foreign Application Priority Data
Nov. 19, 2021 (CN) 202111397963.6

(51) Int. Cl. A61B 5/00; A61L 26/00

(52) U.S. Cl. CPC A61B 5/445; A61L 26/008; A61L 26/0066; A61L 26/0061; A61L 2300/104; A61L 2300/442; A61L 2300/404; A61L 2300/236

(57) ABSTRACT

A preparation of nano silver/dual modified chitosan antibacterial hydrogel dressing with discoloration effect for wound infection judgement and hydrogel dressing prepared by the same provided. The preparation includes the following: carrying out quaternization water-soluble modification on high molecular weight chitosan, and carrying out catecholamination water-soluble modification to prepare dual water-soluble modified chitosan; reducing silver nitrate by using the dual water-soluble modified chitosan to prepare a nano silver/dual water-soluble modified chitosan solution; adding the dual water-soluble modified chitosan, the nano silver/dual water-soluble modified chitosan, curcumin, zwitterion, acrylamide, a cross-linking agent and an initiator into deionized water in sequence, mixing and stirring uniformly, centrifugal de-foaming and casting, to prepare a nano silver/dual modified chitosan antibacterial color-changing hydrogel dressing by one-pot in-situ radical polymerization. The nano silver/dual modified chitosan antibacterial hydrogel dressing exhibits great potential for application in fields such as intelligent indicative wound repair hydrogel dressing and the like.

### CROSS-REFERENCE TO RELATED APPLICATIONS

The present application is a National Stage of International Application No. PCT/CN2022/117686, filed on Sep. 8, 2022, which claims priority to Chinese Patent Application No. 202111397963.6, filed on Nov. 19, 2021, both of which are hereby incorporated by reference in their entireties.

### TECHNICAL FIELD

The present application belongs to the field of medical dressings, in particular to a preparation of nano silver/dual modified chitosan antibacterial hydrogel dressing with discoloration effect for wound infection judgement and hydrogel dressing prepared by the same.

### BACKGROUND

Wound generally refers to the destruction of skin integrity and loss of normal tissue after damage caused by internal or external factors. The common wounds include burn wounds, electric injury wounds, chemical corrosion wounds and the like. Since the wound is in direct contact with the external environment and is highly susceptible to infection, there is an urgent need for wound care to develop advanced wound dressing with antibacterial function and healing promoting effect. Chitosan, as a natural polymer, whose reserves ranks only second to that of cellulose in nature. Furthermore, Chitosan has excellent biocompatibility, hemostasis, antibacterial and wound healing ability, attracting great attention of researchers. However, the low molecular weight chitosan subjected to degradation treatment is inferior in mechanical properties and unstable in physicochemical properties, which makes it difficult to meet practical use requirements. While commercial degraded chitosan is expensive, it is difficult to obtain degraded chitosan products of the same molecular weight even if the chitosan degradation parameters of each batch are strictly controlled. The high molecular weight chitosan of raw material grade has excellent mechanical properties, stable physicochemical properties, and is inexpensive. But how to make high molecular weight chitosan dissolved is currently a major factor restricting its use. Therefore, the water-soluble modification of chitosan is of great interest for expanding the application fields of high molecular weight chitosan, among which quaternization modification of chitosan is the most widely researched. The quaternary ammonium group can also impart antibacterial properties to chitosan. In addition, catecholamination may also increase the water solubility of chitosan, and the catechol structure may also provide potential for functionalization of materials.

Nano silver is a common broad-spectrum antibacterial agent, which has good antibacterial effect against both Gram-positive bacteria and Gram-negative bacteria. However, nano silver tends to have some cytotoxicity, so it is important to find the balance. The above-mentioned quaternized chitosan can form a synergistic antibacterial effect with the nano silver, which can effectively reduce the cytotoxicity of nano silver via cutting down the working concentration while ensuring the antibacterial effect.

However, most of the hydrogel dressings reported or marketed so far have only antibacterial or bacteriostatic properties, with no indication of actual wound infection. Physicians and patients cannot directly access information about the recovery of wound infection, thereby causing great inconvenience in use.

Therefore, a chitosan-based hydrogel dressing with high biocompatibility, antibacterial property and wound infection indicating function is contemplated and designed in the present application. The dressing can satisfy the demands of a complicated use environment, improve the ease of use for patients, and promote the efficiency of wound healing.

### SUMMARY

It is an object of the present application to solve the problems that the existing hydrogel wound dressing has low antibacterial efficiency and no indication function of wound infection. A novel intelligent discoloration chitosan antibacterial hydrogel dressing is prepared by performing dual modification of quaternization and catecholamination on large molecular weight chitosan molecules, combined with the synergistic antibacterial effect of quaternized chitosan, nano silver and curcumin, and the anti-fouling ability and skin-adhesiveness of zwitterion.

The present application is specifically realized by the following steps:

1) dispersing a powder raw material of the high molecular weight chitosan in deionized water with stirring, and adding 0.1-5 wt % acetic acid to prepare a 0.1-1 wt % chitosan aqueous solution; adding glycidyl trimethylamine chloride to the chitosan aqueous solution, reacting for 8 h to prepare quaternized water-soluble modified chitosan, and obtaining a quaternized water-soluble modified chitosan material after dialysis and lyophilization;

2) dissolving the quaternized water-soluble modified chitosan obtained in step 1) in deionized water to prepare an aqueous solution with a mass concentration of 0.1-1 wt %, sequentially adding 3-(2,4-dihydroxy) phenylpropionic acid and 1-ethyl-(3-dimethylaminopropyl) carbodiimide hydrochloride, and adjusting pH within 4-5.5 by using acetic acid throughout; further reacting for 12 h to obtain catecholamination-modified dual water-soluble modified chitosan, and obtaining the dual water-soluble modified chitosan after dialysis and lyophilization, where pH is adjusted within 4-5.5 by acetic acid during the dialysis;

3) dissolving the water-soluble modified chitosan obtained in step 2) in deionized water, adding silver nitrate, reacting in the dark for 0.5 h by virtue of the reduction of the catechol side chain, to prepare a nano silver/dual water-soluble modified chitosan solution; and

4) dissolving the dual water-soluble modified chitosan prepared in step 2), the nano silver/dual water-soluble modified chitosan prepared in step 3), curcumin, zwitterion, acrylamide, the cross-linking agent and the initiator in deionized water in sequence, mixing and stirring uniformly, casting into a mold after centrifugal defoaming, and reacting at 60° C. for 4 h by an one-pot free radical polymerization method to prepare a hydrogel dressing with a nano-composite and semi-interpenetrating polymer network structure.

In the above technical solution, further, the high molecular weight chitosan has a deacetylation degree of 70%-90%, and a molecular weight of 0.5 million-3 million; the dual water-soluble modified chitosan has a quaternization degree of 10%-60%, a catecholamination degree of 5%-60%, and a solubility of 25 mg/mL or more.

Further, the concentration of the glycidyl trimethylamine chloride in step 1) is 0.025-0.10 g/ml.

Further, the concentrations of the 3-(2,4-dihydroxy) phenylpropionic acid and 1-ethyl-(3-dimethylaminopropyl) carbodiimide hydrochloride in step 2) are 0.001-0.005 g/ml and 0.001-0.005 g/ml respectively.

Further, the concentrations of the dual water-soluble modified chitosan and silver nitrate in step 3) are 0.015-0.05 g/ml and 0.015-0.05 g/ml respectively.

Further, the zwitterion in step 4) is phosphorylcholine (PC), sulfobetaine methacrylate (SBMA) or carboxybetaine methacrylate (CBMA); the cross-linking agent is N,N-methylene bisacrylamide (MBAA); the initiator is ammonium persulfate, sodium persulfate or potassium persulfate; where, the concentrations of each components are as follows: 0.05-0.1 g/ml of the water-soluble modified chitosan, 0.0002-0.0005 g/ml of the nano silver/dual water-soluble modified chitosan, 0.001-0.005 g/ml of the curcumin, 0.02-0.1 g/ml of the zwitterions, 0.1-0.5 g/ml of the acrylamide, 0.0002-0.0008 g/ml of the cross-linking agent and 0.001-0.004 g/ml of the initiator.

The hydrogel prepared by the present application has the following functions:

The hydrogel dressing in the present application has pH-responsive intelligent color change function, excellent biocompatibility, high stretchability, anti-fouling ability, multi-effect antibacterial property, long-lasting antibacterial property and skin adhesion property.

High molecular weight chitosan has excellent mechanical properties and stable physical and chemical properties. In the present application, the high molecular weight chitosan is innovatively subjected to dual modification of quaternization and catecholamination. The prepared high molecular weight chitosan has good water solubility and can be used for preparing high-strength hydrogel, thus expanding its application range. At the same time, nano silver can be reduced by catechol group; the size of the nano silver is controlled in the range of 5-25 nm; moreover, since there are a lot of residual amino groups in the molecular chain of quaternized chitosan, it has a chelating effect on silver and can act as a stabilizer for nano silver, and the nano silver/dual modified chitosan solution does not precipitate after standing for more than 10 days.

The hydrogel dressing has bacteriostasis and multiple antibacterial effects: zwitterions, upon polymerization, can form a hydrated layer on the surface of the hydrogel, inhibiting the adhesion of bacteria; nano silver, dual water-soluble modified chitosan and curcumin can achieve a synergistic antibacterial function, in which dual water-soluble modified chitosan provides contact antibacterial property, curcumin and nano silver provide an external dissolution release antibacterial property; hydrogel dressings have long-lasting sustained-release antibacterial properties due to physical and chemical interactions with the bulk functional groups of the hydrogel dressing, such as entanglement of high molecular weight chitosan with the bulk molecular chains of the gel dressing (acrylamide and zwitterionic copolymers), interaction between positively charged silver with electron-rich functional groups, and the like.

Hydrogel dressing has a certain adhesion to skin by virtue of the combined action of the catechol structure and zwitterion, allowing the hydrogel to adhere to the wound skin surface and be used without bandage.

The present application innovatively imparts a responsive discoloration indication function to the hydrogel wound dressing, and is expected to be applied in areas such as intelligent indicative wound repair hydrogel dressings. The intelligent color-changing function of the hydrogel dressing is that the color of curcumin changes with the change of pH: when the pH is 5-6 (healthy skin and healed wounds are weakly acidic, and the pH is about 5-6), the hydrogel dressing is bluish yellow; when the pH value is greater than 7 (if the pH value increases and tends to be alkaline, it indicates that infection may have occurred, and the pH value is greater than 7 in the inflammatory stage), the hydrogel dressing is orange, providing a color change visible to the naked eye and directly indicating infection of the wound.

Compared with the prior art, the present application has the following advantages:

1) the present application greatly improves the water solubility of high molecular weight chitosan by dual modification of quaternization and catecholamination. The above chemical modification process solves the problem that chitosan is difficult to dissolve, and expands the use range of chitosan;

2) according to the present application, the catechol structure on the molecular chain of dual water-soluble modified chitosan can be used to prepare nano silver by reduction reaction. At the same time, the dual modified chitosan molecules also have a stable dispersion effect on the prepared nano silver; the particle size of the nano silver is 5-25 nm, and the nano silver/dual modified chitosan solution does not precipitate after standing for more than 10 days; only acetic acid is used in the whole preparation process, thus the generation of silver-containing precipitates is avoided, and the prepared nano silver has a small particle size and a low cytotoxicity;

3) aiming at the application scenario of wound infection, a nano silver/dual modified chitosan antibacterial hydrogel dressing is successfully prepared through careful selection and combination of raw materials and optimization of the preparation formula of the hydrogel. This hydrogel dressing can indicate the wound infection by discoloration effect;

4) the hydrogel dressing in the present application has a certain adhesion to the skin by virtue of the combined action of the catechol structure and zwitterion, so that the hydrogel can adhere to the wound skin surface and be used without fixing with bandage;

5) nano silver/dual water-soluble modified chitosan, dual water-soluble modified chitosan, curcumin, zwitterion, acrylamide and the like are innovatively used in the present application. The complementary and synergistic performance from the various raw materials is achieved by careful design. The hydrogel dressings simultaneously have functions and properties such as anti-fouling, antibacterial, discoloration effect for wound infection, high stretchability, skin-adhesiveness and biocompatibility. In this scenario, polyacrylamide provides high tensile performance, zwitterion provides anti-fouling performance, quaternized chitosan, curcumin and nano silver provide synergistic antibacterial effect, and curcumin provides discoloration effect to indicate wound infection according to changes in wound pH, so that the hydrogel material designed and prepared in the present application is expected to be applied to the fields of intelligent hydrogel dressing for wound repair and the like.

### BRIEF DESCRIPTION OF DRAWINGS

FIG. 1 illustrates the particle size test result and physical drawing of nano silver prepared according to the present application;

FIG. 2 illustrates the stability test result of the nano silver/dual modified chitosan solution prepared according to the present application;

FIG. 3 is a schematic diagram of the discoloration effect of the hydrogel material prepared according to the present application.

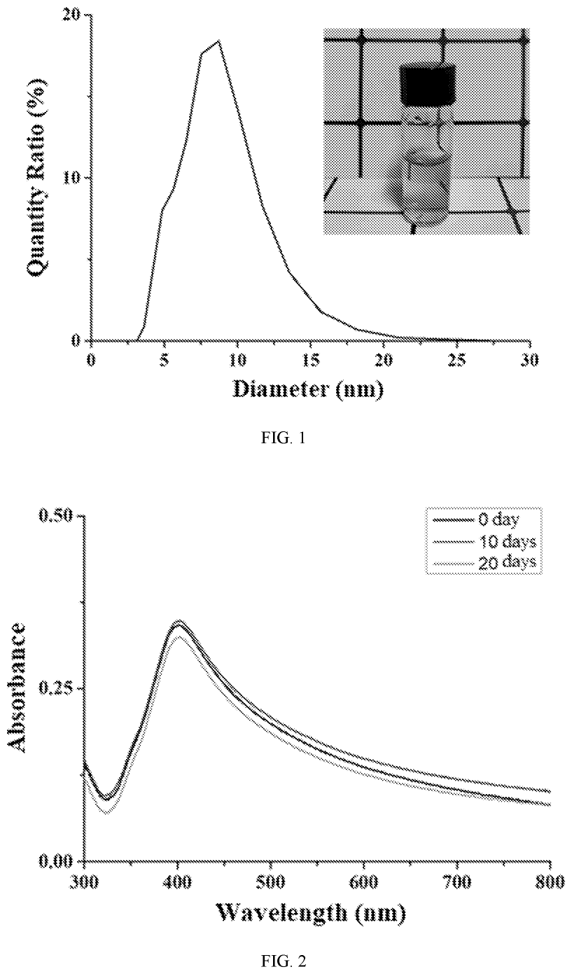

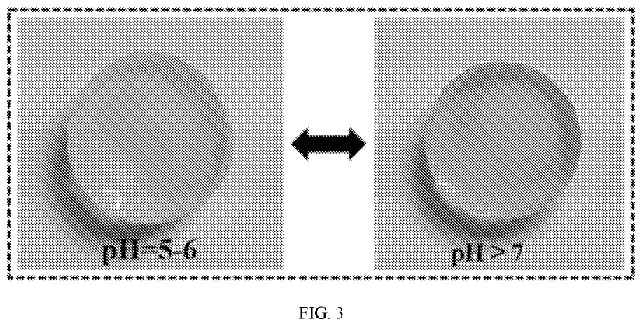

### DESCRIPTION OF EMBODIMENTS

The present application will be further illustrated with reference to examples.

### Example 1

1) High molecular weight chitosan powder of raw material-grade (1 million molecular weight, a deacetylation degree 86%) was dispersed in deionized water with stirring, and 2.5 wt % acetic acid was added to prepare a 0.5 wt % chitosan aqueous solution; glycidyl trimethylamine chloride (0.10 g/ml) was added to the chitosan aqueous solution to react for 8 h to obtain a quaternized water-soluble modified chitosan aqueous solution, which, after dialysis and lyophilization, yielded quaternized water-soluble modified chitosan with a quaternization degree of 60%;

2) the quaternized water-soluble modified chitosan obtained in step 1) was dissolved in deionized water to prepare an aqueous solution with a mass concentration of 0.5 wt %, and 3-(2,4-dihydroxy) phenylpropionic acid (0.0025 g/ml) and 1-ethyl-(3-dimethylaminopropyl) carbodiimide hydrochloride (0.003 g/ml) were added in sequence; the pH of the solution was adjusted to 4-5.5 by acetic acid throughout, and reaction was carried out for 12 h to obtain an aqueous solution of dual water-soluble modified chitosan that was further catecholamination; the dual water-soluble modified chitosan was obtained after dialysis and lyophilization; during dialysis, acetic acid was used to adjust the pH of dialysate to 4-5.5, and the degree of catecholamination of the product was 46%;

3) the dual water-soluble modified chitosan obtained in step 2) was dissolving in deionized water (0.05 g/ml), silver nitrate (0.05 g/ml) was added to react in the dark for 0.5 h to prepare a nano silver/dual water-soluble modified chitosan solution;

4) the dual water-soluble modified chitosan (0.05 g/ml) obtained in step 2), the nano silver/dual water-soluble modified chitosan (0.0005 g/ml), curcumin (0.005 g/ml), SBMA (0.1 g/ml), acrylamide (0.25 g/ml), MBAA (0.0005 g/ml), ammonium persulfate (0.0025 g/ml) and the like were added into deionized water in sequence, mixed and stirred uniformly; after centrifugal de-foaming and casting, and a gel was prepared by one-pot in-situ free radical copolymerization at 60° C. for 4 h;

5) the prepared hydrogel dressing had a tensile strength of 324.6 kPa, an elongation at break of 531%, and an adhesive strength of 8.2 kPa; combined with an ICP-MS test, it was found that the release amount of silver in 7 days was 80%; combined with living dead bacteria staining and antibacterial experiments, it was found that the hydrogel dressing could inhibit the adhesion of bacteria and had good antibacterial effect on Staphylococcus aureus and Escherichia coli models.

### Example 2

1) High molecular weight chitosan powder of raw material-grade (1.8 million molecular weight, a deacetylation degree 74%) was dispersed in deionized water with stirring, and 5 wt % acetic acid was added to prepare a 1 wt % chitosan aqueous solution; glycidyl trimethylamine chloride (0.05 g/ml) was added to the chitosan aqueous solution to react for 8 h to obtain a quaternized water-soluble modified chitosan aqueous solution, which, after dialysis and lyophilization, yielded quaternized water-soluble modified chitosan with a quaternization degree of 34%;

2) the quaternized water-soluble modified chitosan obtained in step 1) was dissolved in deionized water to prepare a quaternized water-soluble modified chitosan aqueous solution with a mass concentration of 0.75 wt %, and 3-(2,4-dihydroxy) phenylpropionic acid (0.005 g/ml) and 1-ethyl-(3-dimethylaminopropyl) carbodiimide hydrochloride (0.003 g/ml) were added in sequence; the pH of the solution was adjusted to 4-5.5 by acetic acid throughout, and reaction was carried out for 12 h to obtain an aqueous solution of dual water-soluble modified chitosan that was further catecholamination; the dual water-soluble modified chitosan was obtained after dialysis and lyophilization; during dialysis, acetic acid was used to adjust the pH of dialysate to 4-5.5, and the degree of catecholamination of the product was 21%;

3) the dual water-soluble modified chitosan obtained in step 2) was dissolving in deionized water (0.03 g/ml), silver nitrate (0.025 g/ml) was added to react in the dark for 0.5 h to prepare a nano silver/dual water-soluble modified chitosan solution;

4) the dual water-soluble modified chitosan (0.05 g/ml) obtained in step 2), the nano silver/dual water-soluble modified chitosan (0.0002 g/ml), curcumin (0.001 g/ml), SBMA (0.04 g/ml), acrylamide (0.5 g/ml), MBAA (0.0002 g/ml), potassium persulfate (0.0035 g/ml) and the like were added into deionized water in sequence, mixed and stirred uniformly; after centrifugal de-foaming and casting, and a gel was prepared by one-pot in-situ free radical copolymerization at 60° C. for 4 h;

5) the prepared hydrogel dressing had a tensile strength of 167 kPa, an elongation at break of 608%, and an adhesive strength of 5.7 kPa; combined with an ICP-MS test, it was found that the release amount of silver in 7 days was 64%; combined with living dead bacteria staining and antibacterial experiments, it was found that the hydrogel dressing could inhibit the adhesion of bacteria and had good antibacterial effect on Staphylococcus aureus and Escherichia coli models.

### Example 3

1) High molecular weight chitosan powder of raw material-grade (2.5 million molecular weight, a deacetylation degree 72%) was dispersed in deionized water with stirring, and 0.1 wt % acetic acid was added to prepare a 0.1 wt % chitosan aqueous solution; glycidyl trimethylamine chloride (0.10 g/ml) was added to the chitosan aqueous solution to react for 8 h to obtain a quaternized water-soluble modified chitosan aqueous solution, which, after dialysis and lyophilization, yielded quaternized water-soluble modified chitosan with a quaternization degree of 56%;

2) the quaternized water-soluble modified chitosan obtained in step 1) was dissolved in deionized water to prepare a quaternized water-soluble modified chitosan aqueous solution with a mass concentration of 0.1 wt %, and 3-(2,4-dihydroxy) phenylpropionic acid (0.001 g/ml) and 1-ethyl-(3-dimethylaminopropyl) carbodiimide hydrochloride (0.001 g/ml) were added in sequence; the pH of the solution was adjusted to 4-5.5 by acetic acid throughout, and reaction was carried out for 12 h to obtain an aqueous solution of dual water-soluble modified chitosan that was further catecholamination; the dual water-soluble modified chitosan was obtained after dialysis and lyophilization; during dialysis, acetic acid was used to adjust the pH of dialysate to 4-5.5, and the degree of catecholamination of the product was 7.8%;

3) the dual water-soluble modified chitosan obtained in step 2) was dissolving in deionized water (0.015 g/ml), silver nitrate (0.015 g/ml) was added to react in the dark for 0.5 h to prepare a nano silver/dual water-soluble modified chitosan solution;

4) the dual water-soluble modified chitosan (0.1 g/ml) obtained in step 2), the nano silver/dual water-soluble modified chitosan (0.0005 g/ml), curcumin (0.003 g/ml), SBMA (0.02 g/ml), acrylamide (0.25 g/ml), MBAA (0.0008 g/ml), sodium persulfate (0.0015 g/ml) and the like were added into deionized water in sequence, mixed and stirred uniformly; after centrifugal de-foaming and casting, and a gel was prepared by one-pot in-situ free radical copolymerization at 60° C. for 4 h;

5) the prepared hydrogel dressing had a tensile strength of 138 kPa, an elongation at break of 431%, and an adhesive strength of 4.4 kPa; combined with an ICP-MS test, it was found that the release amount of silver in 7 days was 53%; combined with living dead bacteria staining and antibacterial experiments, it was found that the hydrogel dressing could inhibit the adhesion of bacteria and had good antibacterial effect on Staphylococcus aureus and Escherichia coli models.

What is claimed is:

1. A method for preparing a nano silver/dual modified chitosan antibacterial hydrogel dressing with discoloration effect for judging wound infection, comprising the following: carrying out quaternization water-soluble modification on high molecular weight chitosan, and carrying out catecholamination water-soluble modification to prepare dual water-soluble modified chitosan; reducing silver nitrate by using the dual water-soluble modified chitosan to prepare a nano silver/dual water-soluble modified chitosan solution; adding the dual water-soluble modified chitosan, the nano silver/dual water-soluble modified chitosan, curcumin, zwitterion, acrylamide, a cross-linking agent and an initiator into deionized water in sequence, mixing and stirring uniformly, centrifugal de-foaming and casting, to prepare a nano silver/dual modified chitosan antibacterial discoloration hydrogel dressing by one-pot in-situ radical polymerization.

2. The method for preparing a nano silver/dual modified chitosan antibacterial hydrogel dressing with discoloration effect for judging wound infection according to claim 1, wherein,
the method comprises the following:
1) dispersing a powder raw material of the high molecular weight chitosan in deionized water with stirring, and adding 0.1-5 wt % acetic acid to prepare a 0.1-1 wt % chitosan aqueous solution; adding glycidyl trimethylamine chloride to the chitosan aqueous solution, reacting for 8 h to prepare quaternized water-soluble modified chitosan, and obtaining a quaternized water-soluble modified chitosan material after dialysis and lyophilization;
2) dissolving the quaternized water-soluble modified chitosan obtained in step 1) in deionized water to prepare an aqueous solution with a mass concentration of 0.1-1 wt %, sequentially adding 3-(2,4-dihydroxy) phenylpropionic acid and 1-ethyl-(3-dimethylamino propyl) carbodiimide hydrochloride, and adjusting pH within 4-5.5 by using acetic acid throughout; further reacting for 12 h to obtain catecholamination-modified dual water-soluble modified chitosan, and obtaining the dual water-soluble modified chitosan after dialysis and lyophilization, wherein pH is adjusted within 4-5.5 by acetic acid during the dialysis;
3) dissolving the water-soluble modified chitosan obtained in step 2) in deionized water, adding silver nitrate, and reacting in the dark for 0.5 h to prepare a nano silver/dual water-soluble modified chitosan solution; and
4) dissolving the dual water-soluble modified chitosan prepared in step 2), the nano silver/dual water-soluble modified chitosan prepared in step 3), curcumin, zwitterion, acrylamide, the cross-linking agent and the initiator in deionized water in sequence, mixing and stirring uniformly, casting into a mold after centrifugal defoaming, and reacting at 60° C. for 4 h by a one-pot free radical polymerization method to prepare a hydrogel dressing with a nano-composite and semi-interpenetrating polymer network structure.

3. The method for preparing a nano silver/dual modified chitosan antibacterial hydrogel dressing with discoloration effect for judging wound infection according to claim 2, wherein,
the high molecular weight chitosan has a deacetylation degree of 70% to 90%, and a molecular weight of 0.5 million to 3 million; and
the dual water-soluble modified chitosan has a quaternization degree of 10% to 60%, a catecholamination degree of 5% to 60%, and a solubility of 25 mg/mL or more.

4. The method for preparing a nano silver/dual modified chitosan antibacterial hydrogel dressing with discoloration effect for judging wound infection according to claim 2, wherein,
the concentration of the glycidyl trimethylamine chloride in step 1) is 0.025-0.10 g/ml;
the concentrations of the 3-(2,4-dihydroxy) phenylpropionic acid and 1-ethyl-(3-dimethylamino propyl) carbodiimide hydrochloride in step 2) are 0.001-0.005 g/ml and 0.001-0.005 g/ml respectively;
the concentrations of the dual water-soluble modified chitosan and silver nitrate in step 3) are 0.015-0.05 g/ml and 0.015-0.05 g/ml respectively; and
the zwitterion in step 4) is phosphorylcholine (PC), sulfobetaine methacrylate (SBMA) or carboxybetaine methacrylate (CBMA); the cross-linking agent is N,N-methylene bisacrylamide (MBAA); the initiator is ammonium persulfate, sodium persulfate or potassium persulfate; wherein, the concentrations of each components are as follows: 0.05-0.1 g/ml of the water-soluble modified chitosan, 0.0002-0.0005 g/ml of the nano silver/dual water-soluble modified chitosan, 0.001-0.005 g/ml of the curcumin, 0.02-0.1 g/ml of the zwitterions, 0.1-0.5 g/ml of the acrylamide, 0.0002-0.0008 g/ml of the cross-linking agent and 0.001-0.004 g/ml of the initiator.

5. A nano silver/dual modified chitosan antibacterial hydrogel dressing with discoloration effect for judging wound infection prepared by the method according to claim 1.
